SEQUENCE LISTING (4 sequences)

SEQ ID NO: 1 - protein, 12 aa, Mus musculus
LAIKNDNLVYVY

SEQ ID NO: 2 - protein, 12 aa, Mus musculus
DVISLYNFKHIY

SEQ ID NO: 3 - protein, 11 aa, Mus musculus
TLFAHGRLVFM

SEQ ID NO: 4 - protein, 12 aa, Mus musculus
LVFMFNVGHKKL

(19) United States
(12) Patent Application Publication
(10) Pub. No.: US 2017/0216409 A1
(43) Pub. Date: Aug. 3, 2017

(54) MODULATION OF LAMININ ALPHA-4 IN THE PREVENTION, TREATMENT, AND MANAGEMENT OF METABOLIC SYNDROMES

(71) Applicants: THE UNIVERSITY OF CHICAGO, Chicago, IL (US); ILLINOIS INSTITUTE OF TECHNOLOGY, Chicago, IL (US)

(72) Inventors: Marcella K. Vaicik, Chicago, IL (US); Ronald N. Cohen, Chicago, IL (US); Eric M. Brey, Chicago, IL (US); Karl Tryggvason, Singapore (SG); Jill Thyboll Kortesmaa, Stockholm (SE)

(21) Appl. No.: 15/500,845

(22) PCT Filed: Aug. 5, 2015
(86) PCT No.: PCT/US15/43821

§ 371 Date: Jan. 31, 2017

Related U.S. Application Data
(60) Provisional application No. 62/033,526, filed on Aug. 5, 2014.

(51) Int. Cl. A61K 38/39; C07K 16/18

(52) U.S. Cl. CPC A61K 38/39; C07K 16/18; C07K 2317/76

(57) ABSTRACT

The disclosure provides methods of preventing or treating metabolic syndrome in a subject by administering an effective amount of an inhibitor of laminin α4 expression, laminin α4 activity, or both.

### BACKGROUND

Obesity continues to increase both domestically and abroad, with the World Health Organization reporting that worldwide obesity rates have doubled since 1980. Over one billion adults 20 years of age or older are overweight with an estimated 500 million of these defined as obese. Obesity is a major risk factor for several chronic diseases, contributing to a dramatic increase in morbidity and mortality due to type 2 diabetes, hypertension, heart disease, and other malignancies [1]. These issues with weight control result in a greater than $190 billion annual burden on US healthcare, and it has been suggested that the negative effects of obesity outweighs the positive effects of smoking cessation on the overall health of the population [2].

The ability to modulate adipose expansion and function would be a tremendous benefit to healthcare. Adipocytes are complex endocrine cells involved in insulin sensitivity, feeding behavior, and neuroendocrine function. A number of soluble hormones and growth factors have been shown to have an influence on adipose insulin sensitivity and expansion. However, little attention has been paid to the contribution to this process made by interactions between adipocytes and the extracellular matrix (ECM). Adipocytes are in constant contact with a network of insoluble proteins and polysaccharides known as the ECM. ECM interactions in adipose tissue can be divided into two major categories: (1) interactions with basement membranes (BM), a thin layer sheet surrounding differentiated adipocytes, and (2) interactions with stromal, or interstitial, ECM that occur when adipocytes invade the tissue stroma during adipose expansion. In other tissues, it is well-known that interactions with the ECM can regulate cell growth, differentiation, and migration, and influence tissue development and repair [3-7]. Little is known about the influence of the ECM on adipose tissue, however.

While adipose-ECM interactions have not been studied extensively, there is some evidence that the ECM plays an important role in regulating adipogenesis and adipocyte function [8]. The synthesis and remodeling of ECM molecules is enhanced during adipogenesis [9] [10]. Differentiating preadipocytes degrade the local matrix, invade the surrounding stroma, and then synthesize new ECM components as they mature [11]. The ECM surrounding preadipocytes transitions from fibronectin-rich to laminin-rich during differentiations, primarily through an increase in an α4 chain containing BM protein laminin [12]. In cell culture models, a preadipocyte cell line has been shown to express laminin, entactin and collagen IV during differentiation [13]. Laminins LN-411 and LN-421, laminin isoforms consisting of a triple helix of the α4, β1 or β2 and γ1 chains, are expressed in excess of other isoforms at this time [14]. The α4 chain of laminin is present in the BM surrounding fully differentiated adipocytes and is upregulated during differentiation. The potential importance of the α4 chain laminins in the BM surrounding adipocytes, however, has not been elucidated.

### SUMMARY

The disclosure provides methods for influencing or controlling basement membrane physiology in adipose tissue by providing modulators of laminin α4 expression, and/or activity. Improved ability to control adipose tissue structure and/or function is a major goal of health professionals and is expected to improve the health and quality of life of mammalian subjects, including a large segment of the human population.

One aspect of the disclosure provides a method of preventing or treating metabolic syndrome in a subject comprising administering an effective amount of an inhibitor of laminin α4. The inhibitor of laminin α4 is an inhibitor of laminin α4 activity and/or expression. In various embodiments, the level of laminin α4 is inhibited, i.e., reduced relative to wild-type levels of laminin α4, or the activity of laminin α4 is inhibited, such as by affecting, e.g., disrupting, cellular interactions, or by affecting both the level of expression of laminin α4 and laminin α4 activity. In some embodiments, the metabolic syndrome is insulin deficiency, insulin resistance, type 1 diabetes, type 2 diabetes, hypercholesterolemia, or a lipoatrophy. Embodiments are also contemplated wherein the inhibition of laminin α4 does not deleteriously change the serum level of at least one compound selected from the group consisting of glucose, a free fatty acid, and a triglyceride. Those of skill in the medical arts understand that changes in the serum levels of these compounds are reflective of improving or worsening health conditions, and can readily recognize whether a change is indicative of an improvement or is indicative of the onset or worsening of a metabolic syndrome condition, i.e., is a deleterious change.

In some embodiments of the method, there is no reduction in food intake by the subject, and some embodiments provide a method wherein food intake by the subject exceeds the caloric intake recommendation of the USDA Recommended Dietary Allowance (RDA) or Dietary Reference Intake (DRI) for such a subject. The disclosure also comprehends embodiments wherein there is a reduction in the level of serum cholesterol in the subject. In some embodiments, the insulin level is lowered. In some embodiments, there is an increase in brown adipose in a subject following administration of the inhibitor of laminin α4 to the subject. Some embodiments involve an increase in beige adipose in a subject following administration of the inhibitor of laminin α4 to the subject. In some embodiments, there is a decrease in white adipose in a subject following administration of the inhibitor of laminin α4 to the subject.

Another aspect of the disclosure provides a method of preventing or treating metabolic syndrome in a subject comprising administering an amount of an inhibitor of laminin α4 effective to alter the thickness of an adipose tissue basement layer. The inhibitor of laminin α4 is an inhibitor of laminin α4 activity and/or expression. In some embodiments, the method results in a thinner basement layer in adipose tissue, while in other embodiments, the basement layer of adipose tissue is thickened by the method.

Still another aspect is drawn to a method of preventing or treating metabolic syndrome in a subject comprising administering an amount of an inhibitor of laminin α4 effective to alter the composition of an adipose tissue basement layer, such as by increasing the relative weight ratio of beige:white adipose, increasing the relative weight ratio of brown:white adipose tissue, or decreasing the relative weight ratio of either beige or brown adipose to white adipose.. The inhibitor of laminin α4 is an inhibitor of laminin α4 activity and/or expression.

Yet another aspect of the disclosure is a method of preventing or treating metabolic syndrome in a subject comprising administering an amount of an inhibitor of laminin α4 (i.e., an inhibitor of laminin α4 activity, and/or laminin α4 expression) effective to inhibit adipogenesis. In various embodiments, any one or more known inhibitor of laminin α4 activity and/or expression, or an inhibitor of α4 activity and/or expression disclosed herein, is contemplated. In some embodiments, a combination of two or more inhibitors of laminin α4 activity or expression is administered to a subject in a mixture, in separate but simultaneous administrations, or in temporally distinct administrations. Also contemplated are embodiments in which at least one inhibitor of laminin α4 activity is administered in combination with at least one inhibitor of laminin α4 expression, with the inhibitors being combined for administration or administered separately to a subject.

Still another aspect of the disclosure is a method of decreasing the rate of increase in adipose tissue volume in a subject comprising administering to the subject an effective amount of an inhibitor of laminin α4 (i.e., an inhibitor of laminin α4 activity and/or expression). As for the preceding aspects of the disclosure, embodiments are contemplated that comprise administering one or more inhibitors of laminin α4 activity and/or one or more inhibitors of laminin α4 expression, with the inhibitors being combined or administered separately in those embodiments involving a plurality of inhibitors.

An aspect of the disclosure is drawn to a method of reducing or preventing liver steatosis in a subject comprising administering to the subject an effective amount of an inhibitor of laminin α4 (i.e., an inhibitor of laminin α4 activity and/or expression). As for the preceding aspects of the disclosure, embodiments are contemplated that comprise administering one or more inhibitors of laminin α4 activity and/or one or more inhibitors of laminin α4 expression, with the inhibitors being combined or administered separately in those embodiments involving a plurality of inhibitors.

Another aspect of the disclosure is a method of inhibiting an increase in serum cholesterol in a subject comprising administering to the subject an effective amount of an inhibitor of laminin α4 (i.e., an inhibitor of laminin α4 activity and/or expression). In some embodiments, the serum cholesterol level is lowered.

For each of the preceding aspects of the disclosure, embodiments are contemplated in which the subject is a human, a farm animal, or a domesticated non-human animal. Embodiments of each aspect of the disclosure are envisioned wherein the subject is a human. Further, embodiments of each aspect are contemplated wherein the inhibitor of laminin α4 is heparin, EDTA, a laminin α4 peptide fragment, an anti-laminin α4 antibody or laminin α4-binding fragment thereof, a lama α4 RNAi, lama α4 siRNA, or a lama α4 miRNA. In some embodiments involving administration of a laminin α4 peptide fragment, the laminin α4 fragment is A4G6 (LAIKNDNLVYVY; SEQ ID NO:1), A4G20 (DVISLYNFKHIY; SEQ ID NO:2), A4G82 (TLFAHGRLVFM; SEQ ID NO:3), or A4G83 (LVFMFNVGHKKL; SEQ ID NO:4). In some embodiments involving administration of an anti-laminin α4 antibody or laminin α4-binding fragment thereof, the anti-laminin α4 antibody or laminin α4-binding fragment thereof is ab69634, ab154314, antibody FC10, antibody BH2, antibody 3D7, antibody 3H2, antibody 5D8, antibody 6A12, antibody 8C10, antibody 9B2,and antibody 839084, or a laminin α4-binding fragment thereof.

Also, some embodiments of the method comprising administration of an anti-laminin alpha 4 antibody or laminin alpha 4-binding fragment thereof are contemplated wherein the anti-laminin alpha 4 antibody or laminin alpha 4-binding fragment thereof is a monoclonal antibody, a humanized antibody; a human antibody; a chimeric antibody; a bispecific or multispecific antibody, an antibody fragment, a Fab, a F(ab′)₂; a Fv; a scFv or single-chain antibody fragment; a diabody; a triabody, a tetrabody, a minibody, a linear antibody; a chelating recombinant antibody, a tribody, a bibody, an intrabody, a nanobody, a small modular immunopharmaceutical (SMIP), a binding-domain immunoglobulin fusion protein, a camelized antibody, a VHH-containing antibody, or a variant or derivative thereof.

In some embodiments of the method, the inhibitor of laminin α4 impedes interaction of laminin α4 with an integrin. In the context of the disclosure, impeding means to inhibit or prevent the interaction of laminin α4 with an integrin. Inhibition or prevention of the interaction of laminin α4 with such exemplary integrins as integrin α6 (CD49f), integrin α4 (CD49d), or integrin β1 (CD29) are contemplated. Exemplary inhibitors of the interaction of laminin α4 with an integrin include GoH3, PS/2, or HA2/5.

Other features and advantages of the disclosure will be better understood by reference to the following detailed description, including the drawing and the examples.

### BRIEF DESCRIPTION OF THE DRAWING

FIG. 1. Immunostaining of whit adipose tissue (WAT). The capillaries and basement membranes (BMs) of adipocytes of control and mutant mice were positive with antibodies against laminin α2 (A, B). Laminin α4 antibodies stained both adipocytic BMs and capillaries in controls (C), while no staining was seen in mutants (D). The laminin α5 antibody(E, F) stained capillaries in mutants and controls, but not the adipocytic BM. Antibodies against type IV collagen (G; H), nidogen (I, J) and perlecan (K, L) all stained both capillaries and adipocytic BMs. Laminin α1 staining was completely negative. The adipocytic BM of the Lama4^{-/-} mice appeared slightly thickened. Bar 100 μm.

FIG. 2. The weight of 8 male Lama4^{-/-} mice housed with 10 littermate heterozygote or wild-type controls revealed a significant difference in weight at the age of 15 weeks (P=0.003), although the difference was not striking until 30-40 weeks of age (A). When fed a high-fat diet (B), male control mice developed obesity earlier, while mutant mice were resistant also in this case (P=0.03 at week 8). The weekly intake of food related to the weight of the animals was not significantly different (P>0.3, mean±SEM, n=3-4 cages, 4 animals per cage) (C).

FIG. 3. The Lama4^{-/-} mice had reduced fat depots. The epididymal fat is delineated in an opened 10-month-old male control mouse (A) and a male Lama4^{-/-} mouse is shown in (B).

FIG. 4. DXA/Image analysis of fat content in representative twelve-month-old male Lama4^{-/-} and littermate control mice fed standard diet. Areas with more than 50% fat are shown as white areas (called DXA fat), while areas with lean mass and bone are shown as black areas (A). Quantification of total fat content in mice, as measured by DXA, showed fat content to be drastically reduced in Lama4^{-/-} mice (B). Computerized tomography (CT) images through the abdomen clearly demonstrated the difference in the amount of fat (C). The thin structure marked with a P in the heterozygote animals designates the peritoneum, black arrows indicate subcutaneous fat, and white arrows indicate intraperitoneal fat.

FIG. 5. Normalized liver mass as a percent of total animal mass of 12-month liver in Lama4^{-/-} animals and control. On high fat diet n=9, on standard diet n=9 for control and n=6 for Lama4^{-/-}. Animals on high fat diet had significantly lower liver mass in Lama4^{-/-} animals (P<0.05).

FIG. 6. Histology of livers of 10-month-old male animals. In control animals on standard diet, only mild accumulation of fat (steatosis) was occasionally seen (A). On the high-fat diet, however, the steatosis was frequently severe (B). In Lama4^{-/-} animals, the histological picture was without signs of steatosis on standard diet (C), and on high-fat diet steatosis was very mild (D).

FIG. 7. Normalized fat pad depot mass as a percent of total animal mass. Epididymal fat pad mass in Lama4^{-/-} mice was significantly lower than Lama4^{+/+} mice (p<0.05). Age-matched animal Lama4^{-/-} and Lama4^{+/+}, n=4 for epididymal and subcutaneous. Error bars represent standard error.

FIG. 8. Adipocyte diameter in subcutaneous and epididymal adipose depots for age-matched Lama4^{-/-} and Lama4^{+/+} mice. For epididymal n=4 and subcutaneous n=5 adipocytes, standard error was shown as the error bars. There is a statistical difference between epididymal and subcutaneous adipocytes (P<0.05). Histology images of epididymal and subcutaneous Lama4^{-/-} and Lama4^{+/+} adipocytes, scale bar equals 300 μm.

FIG. 9. Adipocyte cell density in subcutaneous and epididymal adipose depots for age-matched Lama4^{-/-} and Lama4^{+/+} mice. For epididymal n=4 and subcutaneous n=5 adipocyte cell densities, standard error shown as error bars.

FIG. 10. Basal lipogenesis levels in adipocytes from subcutaneous and epididymal adipose in age-matched knockout (KO) and wild-type (WT) animals. Epididymal fat pad lipogenesis is statistically significant between KO and WT (p<0.05). The epididymal and subcutaneous n=4. Error bars represent standard error.

### DETAILED DESCRIPTION

In the experiments disclosed herein, mice with a null mutation of the laminin α4 gene (Lama4^{-/-}) were used to examine a potential role for α4 chain laminins in adipose tissue expansion and function. Weight gain, adipose function and adipose structure were examined in Lama4^{-/-} mice and compared to control animals. The Lama4^{-/-} mice were found to be resistant to age-related and diet-induced obesity, and exhibited a depot-specific change in adipose structure, volume and function.

Differentiated adipocytes are surrounded by a thin BM layer. ECM in general, and BM specifically, have been shown to regulate cell behavior in a number of tissues and organs [3] [4] [5] [6] [7]. However, there is little knowledge of its role in adipocyte behavior. Laminins are assembled at the cell surface as one component of the basement membrane. Cell receptors maintain the laminins at the cell surface, mediate basement membrane assembly and regulate intracellular signals. Laminin-cell interactions are primarily dependent on the C-terminus domain of the specific laminin a chain. The laminins interact with integrins, dystroglycan, or sulfated glycolipids on the cell surface. Integrins are heterodimeric cell surface receptors consisting of α and β subunits (not to be confused with the α and β chains of laminin). The α4 chain of laminin has been shown to bind to integrins α6β1 and α7β1 in a number of cells. In endothelial cells, laminin α4 signals through the β1 integrin. Altering interactions between laminin α4 and integrins, dystroglycan, or sulfated glycolipids provides an indirect approach to modulate signaling and enhance beiging or adipose metabolic function. In the studies disclosed herein, mice completely lacking a specific BM protein, laminin α4, were used to investigate its role in adipose tissue. Using these mice, a profound influence of laminin α4 on the expansion and function of adipose tissue was found.

Lama4^{-/-} mice were found to gain weight at a much slower rate than control mice. This occurred during normal aging and was more pronounced on a high fat diet. In fact, Lama4^{-/-} mice did not exhibit any differences in weight whether on normal chow or a high fat diet. Differences from wild type animals did not result from reduced food intake, and a reduction in adipose was observed both grossly and via CT scans in the mice deficient in laminin α4. While these results clearly showed that Lama4^{-/-} mice have reduced adiposity, it was initially not clear if this resulted in normal or impaired adipose function. Under both normal and high fat diets, livers in the Lama4^{-/-} mice exhibited little steatosis, suggesting that the decreased body fat did not represent a form of lipodystrophy. Serum profiles of the Lama4^{-/-} mice on normal chow did not indicate severe metabolic defects, as there were no significant changes in the levels of glucose, free fatty acids, triglycerides or cholesterol. Leptin levels were lower in the knockout mice, which was expected because levels are proportional to adipose tissue mass. A high-fat diet resulted in elevated levels of insulin and leptin in control and knockout animals compared to standard diet. The same parameters were also elevated in the Lama4^{-/-} mice fed the high-fat diet.

Laminin α4 is present around cells in the kidney, vasculature and muscle and has been shown to regulate different cell behaviors [22-31]. In order to further evaluate the specific role of laminin α4, adipocytes from these tissues were isolated and their metabolic function was analyzed. Surprisingly, decreased laminin α4 was found to lead to impaired adipocyte lipogenesis. Insulin-stimulated lipogenesis resulted in increased lipogenesis in adipocytes from both Lama4^{-/-} and control animals indicating that insulin responsiveness appears to be intact, but basal lipogenesis levels were lower in Lama4^{-/-} mice. Interestingly, this difference was depot-specific. Adipocytes isolated from epididymal adipose exhibited impaired lipogenesis compared to controls while the subcutaneous adipose depot had similar lipogenesis to control. This depot-specific impairment is further supported by the fact that epididymal adipose depots in Lama4^{-/-} mice are significantly smaller in volume than control animals at about 3.5 months. This difference was observed prior to any measurable differences in total weight. While the total amount of epididymal adipose in Lama4^{-/-} mice was lower than controls, the cells from Lama4^{-/-} mice were larger than cells from control mice. The subcutaneous depots were similar in both total volume and cell size in comparisons between Lama4^{-/-} and control mice. Overall, these results indicate an impaired function in adipose tissue from mice lacking laminin α4 and this impairment is manifested primarily in the epididymal depot.

The experimental studies disclosed herein showed a profound role for laminin α4 on adipose expansion and function. The mechanism underlying this influence, however, has been unclear. There is very little knowledge of the role of the ECM on adipocyte behavior. Cell culture and tissue engineering studies have shown that ECM substrata influence adipocyte behavior. In 2D culture “laminin” was found to be more potent than type IV collagen, fibronectin or type I collagen at promoting differentiation of preadipocytes [32]. This study did not identify which specific laminin isoform was used, but it is likely that this commercial laminin was purified from a mouse soft tissue tumor that has been shown to contain laminin 111 (α1:β1:γ1). Studies with laminin α4 are less common, in part because it only recently became available commercially. Preadipocytes have been shown to produce LN-411 (α4:β1:γ1) during induction to an adipocyte phenotype [14] and ECM mixtures isolated from adipose tissue that are rich in laminin α4 promote greater adipogenesis than ECM from other tissues [33,34]. α4 chain laminin could directly regulate adipocyte function through modulation of cell receptor signaling.

Laminins containing the α4 chain could also influence adipocyte behavior based on indirect effects on the local cell microenvironment. The BM structure appeared thicker in immunohistochemical stains and previous studies have shown that the vascular BM in Lama4^{-/-} mice is altered structurally [15]. This structural change could reflect altered mechanical properties that may influence adipocyte function [35]; Shoham, 2012, Mechanotransduction in adipocytes}. In addition, LN-411, which contains the α4 chain, has a chondroitin sulfate chain [36,37] which may function to sequester growth factors in the vicinity of a cell. This contributes to the regulation of growth factor availability and signaling near cell surface receptors [38]. Alterations in the laminin composition could alter the ability of the BM to bind growth factors and control the local concentration of regulatory factors.

As supported by the following examples, the disclosure establishes that Lama4^{-/-} mice exhibited reduced weight gain in response to both age and high-fat diet. The mice had adipose tissue mass and altered function in a depot-specific manner. In particular, epididymal adipose tissue exhibited decreased mass and altered lipogenesis in Lama4^{-/-} mice, but no differences were observed in the subcutaneous depot. The results indicate that: (1) impaired lipogenesis leads to diminished fat mass in Lama4^{-/-} mice; (2) alterations in lipogenesis are adipose tissue depot-specific; and (3) specific ECM components dramatically influence adipose tissue function.

The working examples that follow include Example 1, which provides the materials and methods for the experiments conducted in the ensuing examples, which collectively provide experimental results supporting the claimed subject matter. Example 2 shows the adipose composition of mice genetically deficient in Laminin α4; Example 3 establishes that Lama4^{-/-} mice are resistant to obesity induced by age or diet; Examples 4, 5, and 6 provide results characterizing Lama4^{-/-} mice in terms of food consumption, serum profile (insulin, leptin, IGF-1, glucose, triglyceride, free fatty acids, and cholesterol), and adipose tissue structure, respectively; and Example 7 establishes adipose tissue function in Lama4^{-/-} and control wild-type mice.

### EXAMPLES

### Example 1

### Material & Methods

### Animals, Diets and Housing

The generation of laminin α4 null mice (Lama4) was previously described [15], and that description is incorporated herein by reference. The mice were backcrossed to C57 BL/6 mice (Charles River) for more than 10 generations. Mice were fed a standard diet, or a high-fat diet containing 45 kcal % fat (D12451, Research Diets), beginning at 4 weeks of age. The animals were fed ad libitum and their food was weighed weekly. The mice were given a food refill up to 500 g after each weighing. The amount of food consumed was divided by the number of animals in a cage as an estimate of intake.

All animal procedures were approved by the IACUC at Karolinska Institutet or the University of Chicago. The animals were housed either in mixed cages (two Lama4^{-/-} and two wild-type control animals) or in cages with only Lama4^{-/-} mice or wild-type animals, in order to rule out the possibility that the weight differences observed were due to differences in dominance behavior. No differences were observed due to housing conditions.

### Immunostaining

For immunostaining in mouse tissues, animals at 4 months of age were sacrificed and tissue harvested. Samples were placed in TissueTek® (Sakura) in plastic molds and frozen in isopentane cooled to its freezing point. Cryosections of 8-12 mm in thickness were made at −38 ° C. The sections were allowed to dry for 1 hour at room temperature and then fixed in acetone for 10 minutes before staining, except for antibody to laminin α4, where the sections were additionally treated for 5 minutes in boiling 1M Urea and washed in distilled water.

The antibodies used were anti-nidogen/entactin (MAB 1946, Chemicon), anti-collagen type IV (polyclonal # AB756P, Chemicon), anti-perlecan (clone HK-102, Seikagaku Corp), anti-laminin α1 (clone 198 (35)), anti-laminin α2 (clone 4H8-2), anti-laminin α4 (polyclonal S8 (36)), and anti-laminin α5 (serum 405). Secondary antibodies were FITC- or Cy3-conjugated and purchased from Jackson ImmunoResearch Laboratories, Inc. Tissue sections were examined with a Leica MDRB microscope (Leica) and pictures were taken with a Hamamatsu digital camera with Openlab (Improvision) software. Digital images were further processed with Photoshop 5.0 (Adobe).

Peripheral Computerized Tomography (pCT)

Computerized tomography in 12-month-old mice was performed 5 mm proximal to the crista iliaca (iliac crest), identified by a longitudinal prescan, with the Stratec pCT XCT Research M, software version 5.4B (Norland Medical Systems) operating at a resolution of 70 μm [16] [17].

### Dual X-ray Absorptiometry (DXA)

DXA was used to determine body composition measurements and for measurements of body fat in the 12-month-old mice. DXA measurements were performed with the Norland pDXA Sabre and the Sabre Research software (Version 3.9.2), as previously described [18]. That description is incorporated herein by reference.

### Serum Analyses

Litter matched, (n=10) Lama4^{-/-} and control mice at 10 months of age were used for serum analysis. The animals were fasted for two hours prior to sampling. Blood was taken from the tail vein and allowed to coagulate for 20-30 minutes at room temperature before centrifugation and separation of serum. Serum leptin and insulin levels were measured by radioimmunoassay (Chrystal Chem. Inc.). Free fatty acids (FFA) were measured by an enzymatic colorimetric method (ACS-ACOD; Wako Chemicals USA, Inc.). Insulin-like growth factor-I (IGF-I) levels were measured by double antibody binding IGF binding protein-blocked radioimmunoassay [19]. Serum glucose was determined with the glucose oxidase method [20]. Triglycerides and cholesterol were assayed using commercially available kits and following the manufacturers' instructions (catalog No. 450032, Triglycerides/GB, Roche Diagnostics GmbH, respective catalog No. 2016630, Cholesterol, CHOD-PAP, Boehringer Mannheim GmbH). Animals used for serum and DXA analyses were sacrificed and the livers harvested for histopathological evaluation (10 Lama4^{-/-} and 10 Lama^{+/-} mice on both diets). For histological staining, the tissue samples were fixed in 10% neutral buffered formalin, paraffin-embedded and stained according to standard protocols. Tissue sections were examined with a Leica MDRB microscope (Leica) and pictures were taken with a Hamamatsu digital camera with Openlab (Improvision) software. Digital images were further processed with Photoshop 5.0 (Adobe).

### Adipose Depot Structure

Lama4^{+/+} and Lama4 mice were fed a standard diet. At 3 months of age, mice were sacrificed. Epididymal and subcutaneous fat depots were harvested, and the masses were assessed. Mass of adipose tissues from each depot was normalized to the total individual animal weight the depot was harvested from using equation (1). The normalized % fat pad mass takes into account variation introduced from individual total animal weights.

(fat pad mass/total animal mass)×100=Normalized % fat pad mass (1)

A portion of each fat pad type was then placed in formaldehyde and paraffin embedded. Samples were sectioned and stained with hematoxylin and eosin. Five images were taken with an Axiovert 200 inverted microscope using a 5× objective (1.3 μm/pixel) (Carl Zeiss MicroImaging, Inc., Thornwood, N.Y.) for each fat pad. The images were used to manually measure the diameters of individual adipocytes using AxioVision (Carl Zeiss MicroImaging).

### Lipogenesis

The fat pads were weighed prior to functional analysis with a lipogenesis assay. The assay was performed as described previously [21], and incorporated herein by reference. Briefly, adipocytes were isolated from the harvested fat pads by collagenase digestion and centrifugation. Isolated adipocytes were incubated with radioactive glucose in Krebs-Ringer bicarbonate containing 10 nM insulin and 1% (w/v) BSA. The lipid fraction was extracted and radioactivity in the triglyceride fraction was measured.

### Statistics

Student's t test was used for all statistical analysis (two-tailed), p<0.05 was considered significant.

### Example 2

### Adipose Composition of Lama4^{-/-} Mice

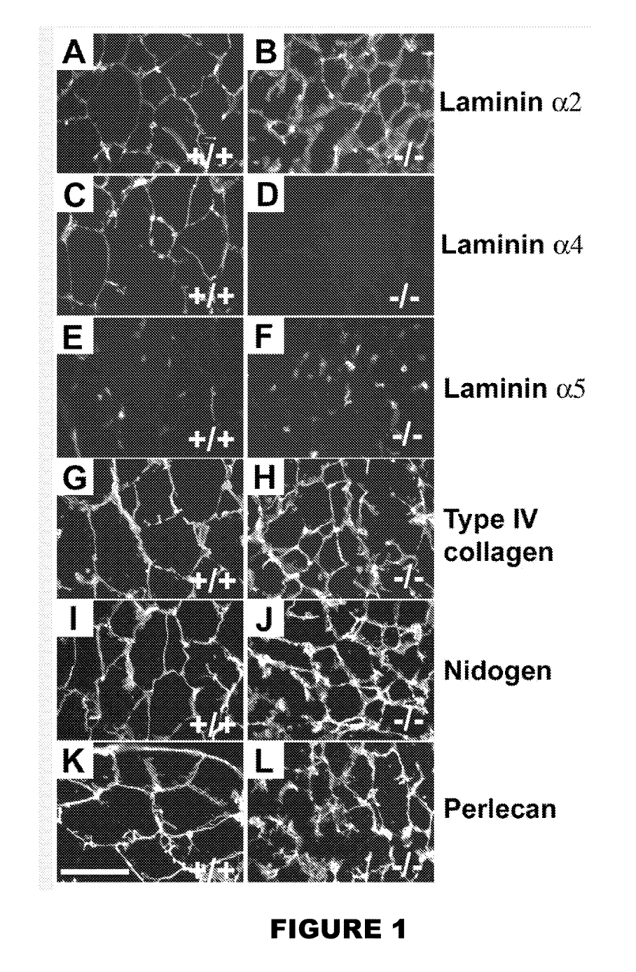

Immunofluorescence staining was first performed to compare BM composition surrounding adipocytes in Lama4^{-/-} and Lama4^{+/+} mice. Staining was performed for known adipose BM proteins, including the α1, α2, α4 and α5 chains of laminin, type IV collagen, nidogen and perlecan. In control mice, the α2 and α4 chains of laminin were present in the BM surrounding mature adipocytes (FIG. 1). Laminin α5 was not observed in mouse adipocyte BM. Type IV collagen, perlecan and nidogen were present in the murine pericellular adipocyte BM. When examining Lama4^{-/-} BM, the only difference in composition was the complete absence of laminin α4. All other BM proteins present in the adipose BM of control mice were also observed in the pericellular BM of Lama4^{-/-} mice. The adipocyte BM appeared somewhat thicker in the Lama4^{-/-} mice.

### Example 3

### Lama4^{-/-} Mice are Resistant to Age- and Diet-Induced Obesity

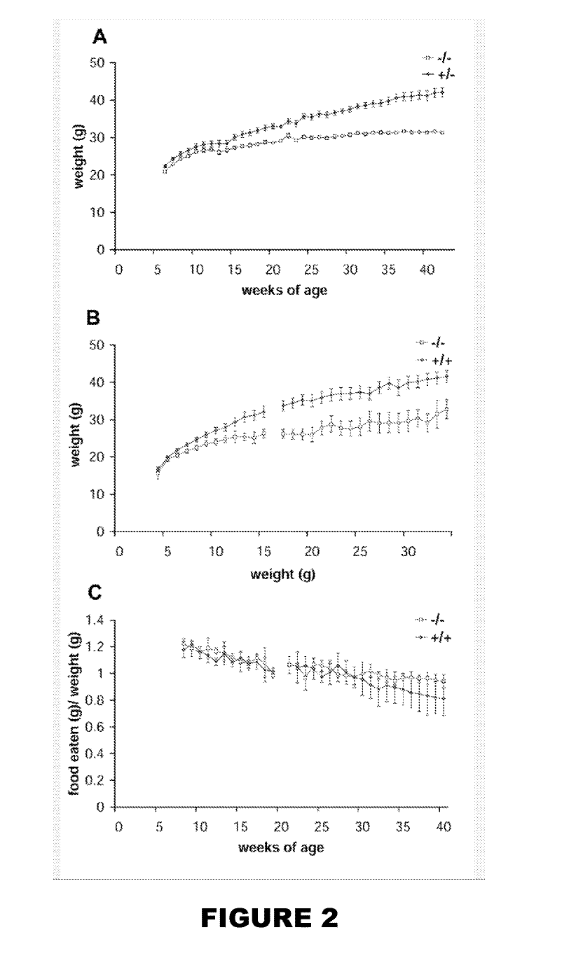

The birth weight of laminin α4 deficient mice was about 10% lower than control littermates, as previously reported [15]. At the time of weaning (6 weeks), no difference in weight was observed between Lama4^{-/-} or control animals (Lama4^{-/-} 20.6±0.32 g, n=8; Lama^{+/-} 22.3±0.44 g, n=10). Animal weights were monitored first on a standard diet. A significant difference in weight was observed by 4 months of age (FIG. 2A; Lama4^{-/-} 27.3±0.48 g, n=8; Lama^{+/-} 30.1±0.76 g, n=10; p=0.003). Lama4^{-/-} weighed less than control animals from this time point and increased steadily over time.

To further investigate weight gain in these animals, mice were supplied a high-fat diet starting at four weeks of age. Lama4^{-/-} animals gained weight at a much lower rate and differences were observed from control animal weights within one month on this diet (Lama4^{-/-} 21.7±0.57 g, n=8; Lama^{+/+} 23.4±0.47 g, n=8; p =0.05, FIG. 2C). This difference continued to increase over time with Lama4^{-/-} exhibiting slow weight gain even on the high-fat diet. In fact, at 12 months of age, there was no difference in the average weight of Lama4^{-/-} mice whether they were on standard or high-fat diets (standard diet 34.3±1.09 versus high-fat diet 35.5±2.12, p=0.6). Control animals rapidly gained weight on the high fat diet resulting in dramatic differences from Lama4^{-/-} at 3.25 months (Lama4^{-/-} 25.4±0.89 g, n=8; Lama^{+/+} 30.7±1.27. g, n=8;p=0.01).

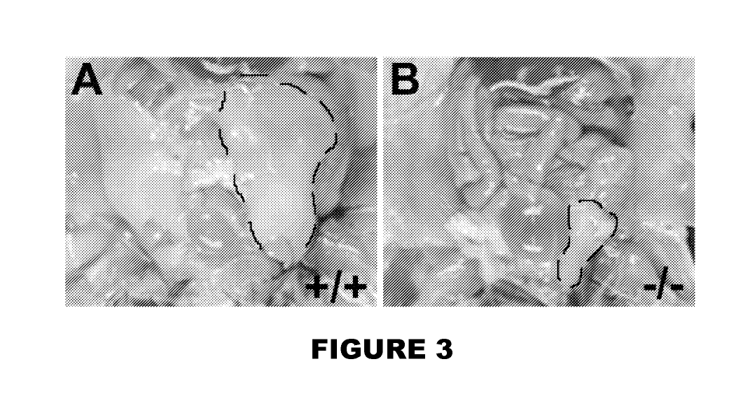

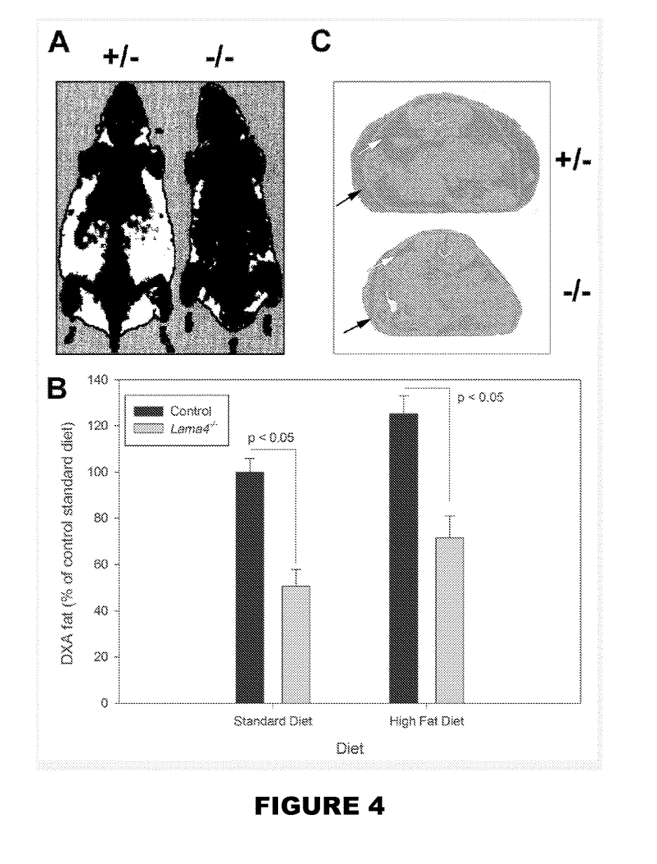

At sacrifice there was a visible difference in the epididymal fat pad volume in Lama4^{-/-} mice compared to controls (FIG. 3). The amount of body fat in the mice was quantified using Dual X-ray Absorptiometry (DXA). Drastically reduced adipose volumes were observed in Lama4^{-/-} mice compared to controls (Standard diet Lama4^{-/-} 50.62±7.29%, n=6; Lama^{+/+} 100.0±5.80%, n=4; p<0.05; High Fat diet Lama4^{-/-} 71.63±9.40%, n=6; Lama 125.24±7.67%, n=8; p<0.05; FIG. 4A, B). An abdominal CT scan of Lama4^{-/-} mice at 12 months further illustrated the fact that the adipose volumes were reduced. As shown in FIG. 3C, at this time point the amounts of both subcutaneous and intra abdominal adipose appeared lower than in littermate controls.

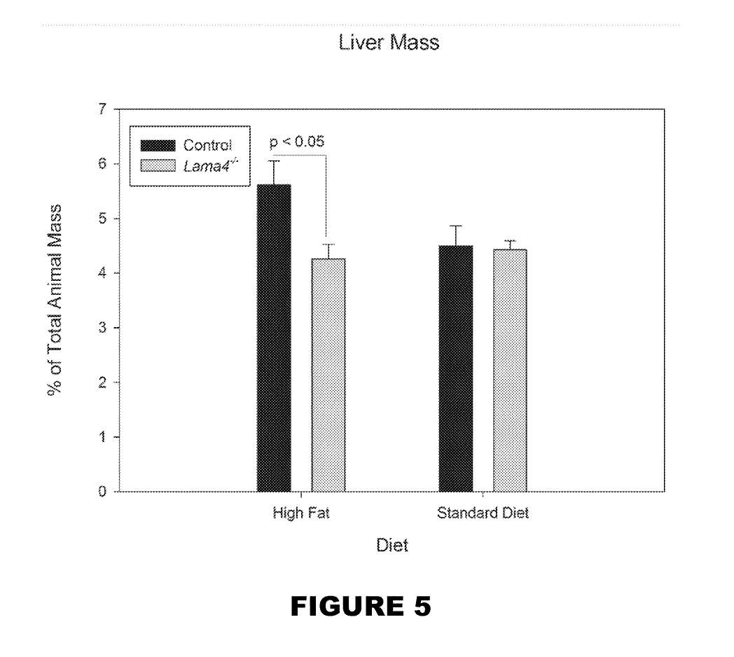

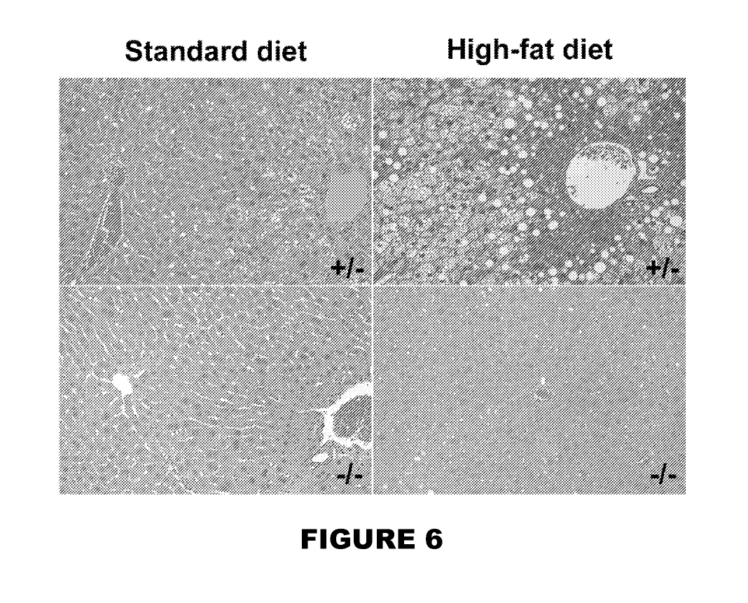

There was no difference in the weights of livers from Lama4^{-/-} and controls on standard diet, but the livers were smaller in Lama4^{-/-} animals on high-fat diet (FIG. 5). The livers of control mice on standard diet showed mild liver steatosis after 12 months, while this was not seen in Lama4^{-/-} mice (FIG. 6A, C) suggesting that the decreased body fat in Lamaα4^{-/-} mice does not represent a form of lipodystrophy. On a high-fat diet, the control mice had moderate to severe steatosis (FIG. 6B), while Lama4^{-/-} mice only had very mild steatosis (FIG. 6D). This decreased steatosis could help explain the smaller mass of livers of Lama4^{-/-} mice relative to controls on the high-fat diet.

### Example 4

### Food Consumption

In order to evaluate whether the observed differences in weight and body composition resulted from lower food consumption, food intake was quantified weekly. There was no difference in the total amount of food consumed between Lama4^{-/-} and control animals (p>0.3 at any time-point, FIG. 1D). These results suggest that the weight differences observed did not result from hypophagia in the Lama4^{-/-} mice.

### Example 5

### Serum Profile

The serum profiles of mice at 10 months of age on normal and high-fat diet were determined. Serum samples were taken from 10-month-old males and heterozygote littermates (n=5-10 in each group, housed 2 Lama4^{-/-} and 2 littermate Lama4^{+/-} in each cage) after two hours of fasting. The levels of leptin, glucose, insulin, insulin-like growth factor-1 (IGF-1), free fatty acids (FFA), triglycerides (TG) and cholesterol were measured. The results are shown in Table 1, where *, P<0.05; ** P≦0.01; ***, P≦0.001. As revealed by the data in Table 1, serum levels of insulin, leptin, and IGF-1 were decreased in Lama4^{-/-} mice, while serum levels of glucose, triglyceride, and free fatty acids were unchanged. Interestingly, Lama4^{-/-} animals had reduced serum cholesterol compared to controls, even on a high-fat diet.

TABLE 1
Clinical Chemical Analysis

| | Standard diet | | High-fat diet | |
|---|---|---|---|---|
| Serum analyses | Lama4^{+/-} | Lama4^{-/-} | Lama4^{+/-} | Lama4^{-/-} |
| Leptin (pg/ml) | 7079 ± 2085 | 759 ± 339** | 44969 ± 3253 | 16489 ± 4886*** |
| Glucose (mg/dL) | 147 ± 9 | 132 ± 8 | 246 ± 13 | 250 ± 15 |
| Insulin (pg/ml) | 1229 ± 245 | 562 ± 31* | 8566 ± 955 | 4249 ± 873** |
| IGF-1 (ng/ml) | 414 ± 23 | 327 ± 10** | 415 ± 24 | 348 ± 18* |
| FFA (mEq/l) | 0.39 ± 0.02 | 0.57 ± 0.11 | 0.51 ± 0.07 | 0.55 ± 0.11 |
| TG (mmol/l) | 0.76 ± 0.05 | 0.65 ± 0.07 | 0.81 ± 0.05 | 0.73 ± 0.05 |
| Cholesterol (mmol/l) | 2.52 ± 0.08 | 2.32 ± 0.05 | 5.98 ± 0.42 | 3.43 ± 0.34*** |

### Example 6

### Adipose Tissue Structure

Lama4^{-/-} and age matched control animals 13 to 15 week on a standard diet were used to further examine adipose structure and function. This time range was selected because it is prior to any statistically significant weight differences between Lama4^{-/-} and control animals, allowing for the examination of adipose function without confounding results due to obesity. At the time of sacrifice there were no differences in total animal mass between Lama4^{-/-} and control animals (Lama4^{-/-} 25.93±0.77 g, n=4; Lama^{+/+} 26.60±2.35 g, n=4; p=0.80).

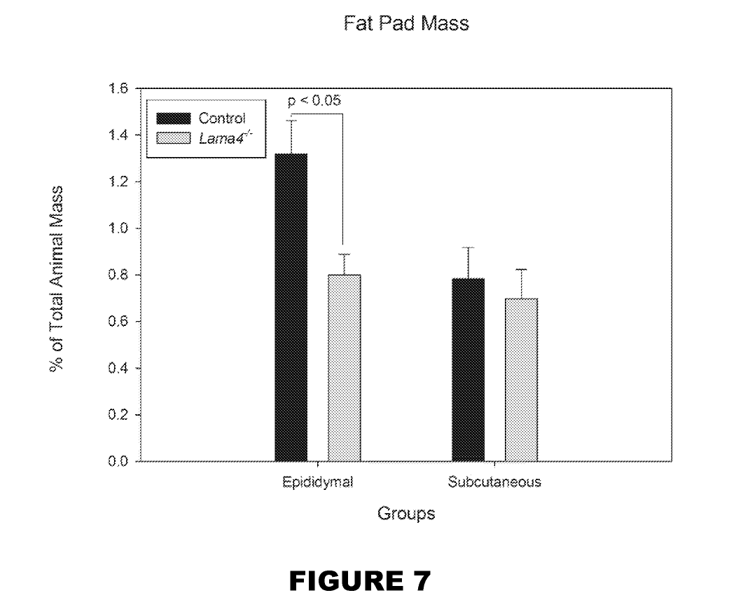

Epididymal and subcutaneous adipose tissue were harvested from the mice. The normalized percentage mass of Lama4^{-/-} mice's epididymal adipose was significantly less than control mice (Lama4^{-/-} 0.80±0.09% , n=4; Lama^{+/+} 1.32±0.14%, n=4; p=0.022) (FIG. 7). The mass of Lama4^{-/-} mice's epididymal adipose was less than control mice (Lama4^{-/-} 0.21±0.03 g, n=4; Lama^{+/+} 0.36±0.07 g, n=4; p=0.10). Interestingly, no differences in mass were observed between subcutaneous adipose mass (Lama4 ^{-/-} 0.18±0.04 g, n=4; Lama^{+/+} 0.21±0.06 g, n=4; p=0.69). These results are at a much earlier time point (about 3.5 months) than the DXA results shown above (12 months) when all adipose depots appeared reduced in volume. These results indicate that prior to any phenotypic observations in total animal weight gain, epididymal volume was reduced in Lama4^{-/-} mice.

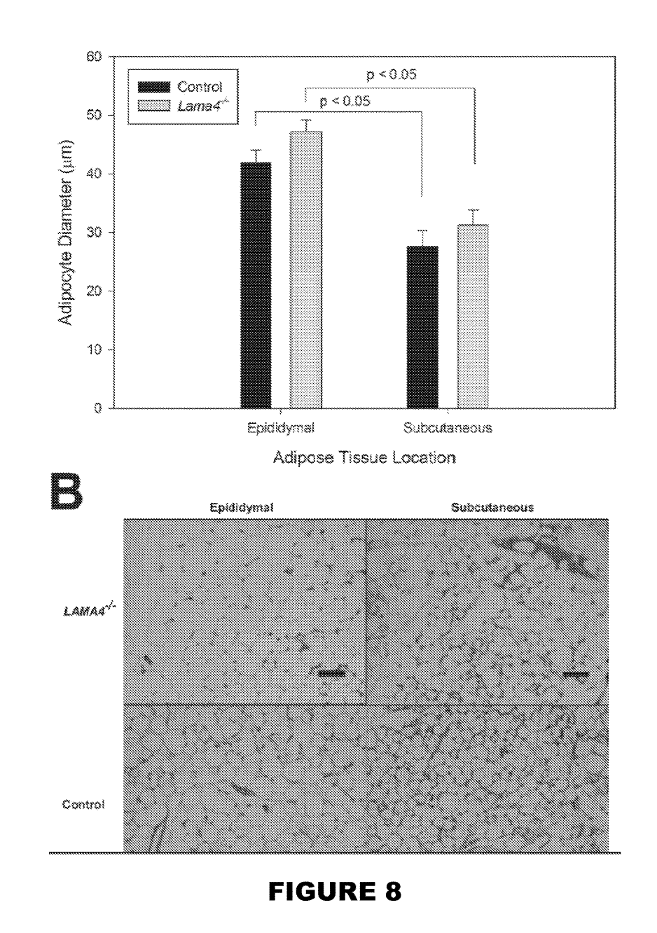

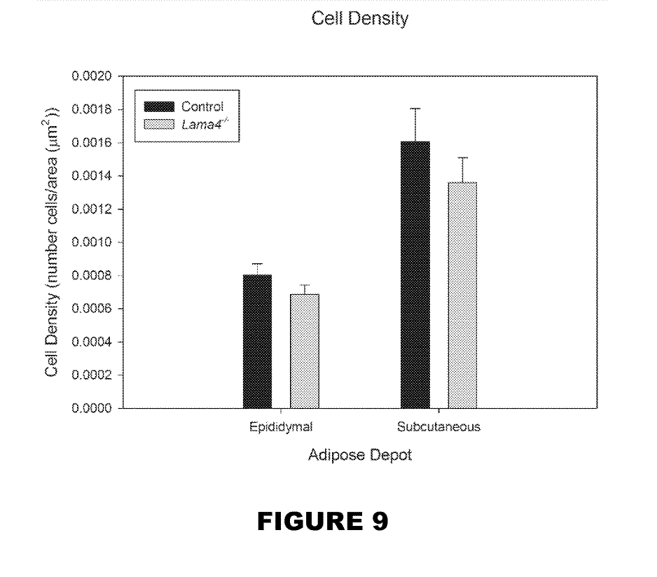

Histomorphometrix analysis was used to further analyze adipose tissue in the different depots. Mean adipocyte size determined from histological stains was greater in epididymal adipose tissue than in the subcutaneous adipose depots in both types of mice. In both subcutaneous and epididymal depots the mean adipose size was greater in the Lama4^{-/-} mice (FIG. 8). Cell density (number of cells per area) was lower in both epididymal and subcutaneous adipose depots for Lama4^{-/-} mice relative to controls (FIG. 9).

### Example 7

### Adipose Tissue Function

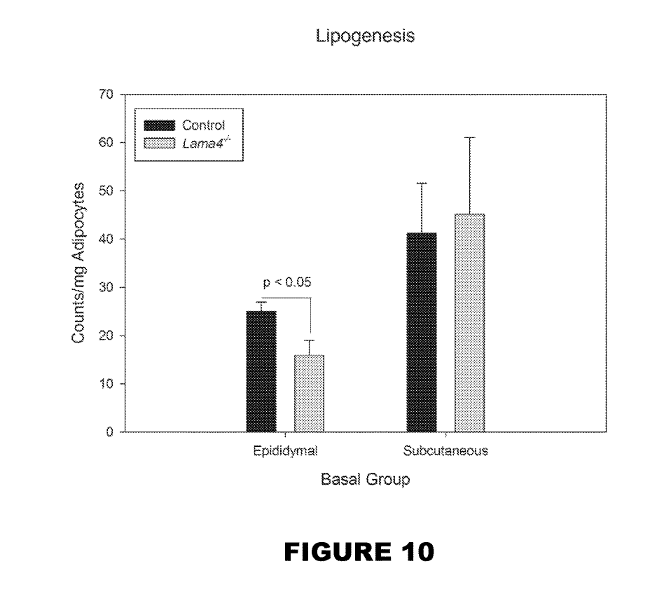

The reduced adiposity may be an indication of altered metabolic function. Adipose function was examined by quantifying basal and insulin-stimulated lipogenesis in adipose isolated from Lama4^{-/-} and control mice. The epididymal depot of Lama4^{-/-} mice exhibited impaired basal lipogenesis levels (FIG. 10). Interestingly, there were no differences in lipogenesis between subcutaneous adipocytes isolated from Lama4^{-/-} and control mice.

### REFERENCES

1. Frayn K N (2000) Visceral fat and insulin resistance—causative or correlative? British Journal of Nutrition 83: S71-S77.

2. Stewart S T, Cutler D M, Rosen A B (2009) Forecasting the effects of obesity and smoking on U.S. life expectancy. N Engl J Med 361: 2252-2260.

3. Aumailley M, Gayraud B (1998) Structure and biological activity of the extracellular matrix. Journal of Molecular Medicine 76: 253-265.

4. Paulsson M (1992) Basement-Membrane Proteins—Structure, Assembly, And Cellular Interactions. Critical Reviews in Biochemistry and Molecular Biology 27: 93-127.

5. Kalluri R (2003) Basement membranes: Structure, assembly and role in tumour angiogenesis. Nature Reviews Cancer 3:422-433.

6. Lin C Q, Bissell M J (1993) Multifaceted Regulation Of Cell-Differentiation By Extracellular-Matrix. Faseb Journal 7: 737-743.

7. Domogatskaya A, Rodin S, Tryggvason K (2012) Functional Diversity of Laminins. Annual Review of Cell and Developmental Biology, Vol 28 28: 523-553.

8. Kawaguchi N, Toriyama K, Nicodemou-Lena E, Inou K, Torii S, et al. (1998) De novo adipogenesis in mice at the site of injection of basement membrane and basic fibroblast growth factor. Proceedings of the National Academy of Sciences of the United States of America 95: 1062-1066.

9. Kawaguchi N, Toriyama K, Nicodemou-Lena E, Inou K, Torii S, et al. (1999) Reconstituted basement membrane potentiates in vivo adipogenesis of 3T3-F442A cells. Cytotechnology 31: 215-220.

10. Chavey C, Mari B, Monthouel M N, Bonnafous S, Anglard P, et al. (2003) Matrix metalloproteinases are differentially expressed in adipose tissue during obesity and modulate adipocyte differentiation. Journal of Biological Chemistry 278:11888-11896.

11. Kubo Y, Kaidzu S, Nakajima I, Takenouchi K, Nakamura F (2000) Organization of extracellular matrix components during differentiation of adipocytes in long-term culture. In Vitro Cellular & Developmental Biology—Animal 36:38-44.

12. Nie J, Sage E H (2009) SPARC Inhibits Adipogenesis by Its Enhancement of beta-Catenin Signaling. Journal of Biological Chemistry 284: 1279-1290.

13. Aratani Y, Kitagawa Y (1988) Enhanced Synthesis And Secretion Of Type-Iv Collagen And Entactin During Adipose Conversion Of 3t3-L1 Cells And Production Of Unorthodox Laminin Complex. Journal of Biological Chemistry 263: 16163-16169.

14. Niimi T, Kumagai C, Okano M, Kitagawa Y (1997) Differentiation-dependent expression of laminin-8 (alpha 4 beta 1 gamma 1) mRNAs in mouse 3T3-L1 adipocytes. Matrix Biology 16: 223-230.

15. Thyboll J, Kortesmaa J, Cao R H, Soininen R, Wang L, et al. (2002) Deletion of the laminin alpha 4 chain leads to impaired microvessel maturation. Molecular and Cellular Biology 22: 1194-1202.

16. Wallenius V, Wallenius K, Ahren B, Rudling M, Carlsten H, et al. (2002) Interleukin-6-deficient mice develop mature-onset obesity. Nature Medicine 8: 75-79.

17. Windahl S H, Vidal O, Andersson G, Gustafsson J A, Ohlsson C (1999) Increased cortical bone mineral content but exchanged trabecular bone mineral density in female ER beta(−/−) mice. Journal of Clinical Investigation 104: 895-901.

18. Sjogren K, Hellberg N, Bohlooly-Y M, Savendahl L, Johansson M S, et al. (2001) Body fat content can be predicted in vivo in mice using a modified dual-energy X-ray absorptiometry technique. Journal of Nutrition 131: 2963-2966.

19. Blum W F, Breier B H (1994) Radioimmunoassays for IGFs and IGFBPs. Growth Regul 4 Suppl 1: 11-19.

20. Bruss M L, Black A L (1978) Enzymatic microdetermination of glycogen. Anal Biochem 84: 309-312.

21. Neel B A, Brady M J, Sargis R M (2013) The Endocrine Disrupting Chemical Tolylfluanid Alters Adipocyte Metabolism via Glucocorticoid Receptor Activation. Molecular Endocrinology 27: 394-406.

22. Sorokin L M, Pausch F, Durbeej M, Ekblom P (1997) Differential expression of five laminin alpha (1-5) chains in developing and adult mouse kidney. Developmental Dynamics 210: 446-462.

23. Ringelmann B, Roder C, Hallmann R, Maley M, Davies M, et al. (1999) Expression of laminin alpha 1, alpha 2, alpha 4, and alpha 5 chains, fibronectin, and tenascin-C in skeletal muscle of dystrophic 129ReJ dy/dy mice. Experimental Cell Research 246: 165-182.

24. Petajaniemi N, Korhonen M, Kortesmaa J, Tryggvason K, Sekiguchi K, et al. (2002) Localization of laminin alpha 4-chain in developing and adult human tissues. Journal of Histochemistry & Cytochemistry 50: 1113-1130.

25. Patton B L, Miner J H, Chiu A Y, Sanes J R (1997) Distribution and function of laminins in the neuromuscular system of developing, adult, and mutant mice. Journal of Cell Biology 139: 1507-1521.

26. Miner J H, Patton B L, Lentz S I, Gilbert D J, Jenkins N A, et al. (1997) The laminin alpha chains: Expression, developmental transitions, and chromosomal locations of alpha 1-5, identification of heterotrimeric laminins 8-11, and cloning of a novel alpha 3 isoform. Journal of Cell Biology 137: 685-701.

27. Gu Y C, Sorokin L, Durbeej M, Hjalt T, Jonsson J I, et al. (1999) Characterization of bone marrow laminins and identification of alpha 5-containing laminins as adhesive proteins for multipotent hematopoietic FDCP-mix cells. Blood 93: 2533-2542.

28. Lefebvre O, Sorokin L, Kedinger M, Simon-Assmann P (1999) Developmental expression and cellular origin of the laminin alpha 2, alpha 4, and alpha 5 chains in the intestine. Developmental Biology 210: 135-150.

29. Geberhiwot T, Ingerpuu S, Pedraza C, Neira M, Lehto U, et al. (1999) Blood platelets contain and secrete laminin-8 (alpha 4 beta 1 gamma 1) and adhere to laminin-8 via alpha 6 beta 1 integrin. Experimental Cell Research 253: 723-732.

30. Frieser M, Nockel H, Pausch F, Roder C, Hahn A, et al. (1997) Cloning of the mouse laminin alpha 4 cDNA. Expression in a subset of endothelium. European Journal of Biochemistry 246: 727-735.

31. Sorokin L M, Maley M A L, Moch H, von der Mark H, von der Mark K, et al. (2000) Laminin alpha 4 and integrin alpha 6 are upregulated in regenerating dy/dy skeletal muscle: Comparative expression of laminin and integrin isoforms in muscles regenerating after crush injury. Experimental Cell Research 256: 500-514.

32. Hausman G J, Wright J T, Richardson R L (1996) The influence of extracellular matrix substrata on preadipocyte development in serum-free cultures of stromal-vascular cells. Journal of Animal Science 74:2117-2128.

33. Uriel S, Huang J-J, Moya M L, Francis M E, Wang R, et al. (2008) The role of adipose protein derived hydrogels in adipogenesis. Biomaterials 29: 3712-3719.

34. Uriel S, Labay E, Francis-Sedlak M, Moya M, Weichselbaum R, et al. (2009) Extraction and Assembly of Tissue-Derived Gels for Cell Culture and Tissue Engineering. Tissue Engineering Part C-Methods 15: 309-321.

35. Shoham N, Gottlieb R, Sharabani-Yosef O, Zaretsky U, Benayahu D, et al. (2012) Static mechanical stretching accelerates lipid production in 3T3-L1 adipocytes by activating the MEK signaling pathway. American Journal of Physiology-Cell Physiology 302: C429-C441.

36. Sasaki T, Mann K, Timpl R (2001) Modification of the laminin alpha 4 chain by chondroitin sulfate attachment to its N-terminal domain. Febs Letters 505: 173-178.

37. Kortesmaa J, Doi M, Patarroyo M, Tryggvason K (2002) Chondroitin sulphate modification in the alpha 4 chain of human recombinant laminin-8 (alpha 4 beta 1 gamma 1). Matrix Biology 21: 483-486.

38. Park P W, Reizes O, Bernfield M (2000) Cell surface heparan sulfate proteoglycans: Selective regulators of ligand-receptor encounters. Journal of Biological Chemistry 275: 29923-29926.

Each of the references cited herein is hereby incorporated by reference in its entirety, or in relevant part, as would be apparent from the context of the incorporation.

The disclosed subject matter has been described with reference to various specific embodiments and techniques. It should be understood, however, that many variations and modifications may be made while remaining within the spirit and scope of the disclosed subject matter.

1. A method of preventing or treating metabolic syndrome in a subject comprising administering an effective amount of an inhibitor of laminin α4 to a subject.

2. The method according to claim 1 wherein the metabolic syndrome is insulin deficiency, insulin resistance, type 1 diabetes, type 2 diabetes, hypercholesterolemia, or a lipoatrophy.

3. The method according to either claim 1 or claim 2 wherein the inhibition of laminin α4 does not deleteriously change the serum level of at least one compound selected from the group consisting of glucose, a free fatty acid, and a triglyceride.

4. The method according to either claim 1 or claim 2 wherein there is no reduction in food intake by the subject.

5. The method according to either claim 1 or claim 2 wherein food intake by the subject exceeds the caloric intake recommendation of the USDA Recommended Dietary Allowance (RDA) or Dietary Reference Intake (DRI) for such a subject.

6. The method according to either claim 1 or claim 2 wherein there is a reduction in the level of serum cholesterol in the subject.

7. A method of preventing or treating metabolic syndrome in a subject comprising administering an amount of an inhibitor of laminin α4 effective to alter the thickness of an adipose tissue basement layer.

8. A method of preventing or treating metabolic syndrome in a subject comprising administering an amount of an inhibitor of laminin α4 effective to alter the composition of an adipose tissue basement layer to increase the relative weight ratio of beige or brown adipose to white adipose.

9. A method of preventing or treating metabolic syndrome in a subject comprising administering an amount of an inhibitor of laminin α4 activity effective to inhibit adipogenesis.

10. A method of decreasing the rate of increase in adipose tissue volume in a subject comprising administering to the subject an effective amount of an inhibitor of laminin α4.

11. A method of reducing or preventing liver steatosis in a subject comprising administering to the subject an effective amount of an inhibitor of laminin α4.

12. A method of inhibiting an increase in serum cholesterol in a subject comprising administering to the subject an effective amount of an inhibitor of laminin α4.

13. The method according to claim 12 wherein the serum cholesterol level is lowered.

14. The method according to any of the preceding claims wherein the subject is a human, a farm animal, or a domesticated non-human animal.

15. The method according to claim 14 wherein the subject is a human.

16. The method according to any of the preceding claims wherein the inhibitor of laminin α4 is heparin, EDTA, a laminin α4 peptide fragment, an anti-laminin α4 antibody or laminin α4-binding fragment thereof, a lama α4 RNAi, lama α4 siRNA, or a lama α4 miRNA.

17. The method according to claim 16 wherein the laminin α4 fragment is A4G6 (LAIKNDNLVYVY; SEQ ID NO:1), A4G20 (DVISLYNFKHIY; SEQ ID NO:2), A4G82 (TLFAHGRLVFM; SEQ ID NO:3), or A4G83 (LVFMFNVGHKKL; SEQ ID NO:4).

18. The method according to claim 16 wherein the anti-laminin α4 antibody or laminin α4-binding fragment thereof is ab69634, ab154314, antibody FC10, antibody BH2, antibody 3D7, antibody 3H2, antibody 5D8, antibody 6A12, antibody 8C10, antibody 9B2,and antibody 839084, or a laminin α4-binding fragment thereof.

19. The method according to claim 16 wherein the anti-laminin alpha 4 antibody or laminin alpha 4-binding fragment thereof is a monoclonal antibody, a humanized antibody; a human antibody; a chimeric antibody; a bispecific or multispecific antibody, an antibody fragment, a Fab, a F(ab′)₂; a Fv; a scFv or single-chain antibody fragment; a diabody; a triabody, a tetrabody, a minibody, a linear antibody; a chelating recombinant antibody, a tribody, a bibody, an intrabody, a nanobody, a small modular immunopharmaceutical (SMIP), a binding-domain immunoglobulin fusion protein, a camelized antibody, a VHH-containing antibody, or a variant or derivative thereof.

20. The method according to any one of claims 16-19 wherein the inhibitor of laminin α4 impedes interaction of laminin α4 with an integrin.

21. The method according to claim 20 wherein the integrin is integrin α6 (CD49f), integrin α4 (CD49d), or integrin β1 (CD29).

22. The method according to claim 20 wherein the inhibitor is GoH3, PS/2, or HA2/5.

23. The method according to claim 1 wherein the insulin level is lowered.

24. The method according to claim 1 wherein there is an increase in brown adipose in a subject following administration of the inhibitor of laminin α4 to the subject.

25. The method according to claim 1 wherein there is an increase in beige adipose in a subject following administration of the inhibitor of laminin α4 to the subject.

26. The method according to claim 1 wherein there is a decrease in white adipose in a subject following administration of the inhibitor of laminin α4 to the subject.
